(19) United States
(12) Patent Application Publication
(10) Pub. No.: US 2008/0161883 A1
(43) Pub. Date: Jul. 3, 2008

(54) Nerve Stimulation Apparatus And Method

(75) Inventor: Minogue Conor, Co. Calway (IE)

Correspondence Address: DRINKER BIDDLE & REATH (DC), 1500 K STREET, N.W., SUITE 1100, WASHINGTON, DC, 20005-1209, US

(73) Assignee: BMR Research & Development Limited, Galway (IE)

(21) Appl. No.: 11/909,190

(22) PCT Filed: Mar. 23, 2006
(86) PCT No.: PCT/IB2006/001298

§ 371 Date: Sep. 20, 2007

(30) Foreign Application Priority Data
Mar. 23, 2005 (GB) 0505940.7

(51) Int. Cl. A61N 1/36

(52) U.S. Cl. 607/48

(57) ABSTRACT

The invention relates to nerve stimulation apparatus having a control unit, first and second electrodes which impart an electrical current on the user's body there between and first and second sensors adapted to detect an electrical signal differential detected there between. This allows electrical stimulation of the users muscles to be provided whilst allowing the cardiac cycle of the user to be simultaneously monitored by the apparatus. There is also provided a method of providing nerve stimulation and exercise monitoring.

The present invention relates to nerve stimulation apparatus, and more particularly but not exclusively, relates to nerve stimulation apparatus for stimulating muscles by way of the muscle motor nerve in order to maximise the benefit obtained by muscle stimulation, for example whilst exercising.

Individuals are generally interested in obtaining maximum benefit from a given amount of exertion when exercising. This can currently be facilitated using Electrical Muscle Stimulation (EMS) devices which pass a controlled electrical current through the is motor nerve of targeted muscles (such as the abdominal muscles) in order to cause those muscles to contract. This has the effect of toning those targeted muscles with minimal effort and can be used whilst performing an exercise routine in order to maximise benefit from the routine.

Although EMS devices assist toning of muscles they do not provide any way of ensuring that cardiovascular exercise is optimised.

According to the present invention there is provided nerve stimulation apparatus comprising: —

a control unit;

a first electrode;

a second electrode, said first and second electrodes being adapted to impart an electrical current there between;

a first sensor; and

a second sensor, said first and second sensors being adapted to detect an electrical signal differential detected there between.

Preferably, said first and second electrodes are adapted to provide electrical stimulation of a motor nerve of a user's muscles located adjacent to said first and second electrodes. Optionally, said first and second electrodes are provided adjacent the user's abdominal muscles. Alternatively, said first and second electrodes are provided at a location close to any motor nerve of the user, such as a peripheral motor nerve.

Preferably, said first and second sensors are adapted to monitor a user's heart rate.

Preferably, said second sensor is provided by said first electrode.

Preferably, a third sensor is provided by said second electrode.

Preferably, said first sensor is adapted to be provided at a location that is closer to the user's heart than the or each of the second and third sensors. More preferably, the first sensor is adapted to be attached to the thoracic region of a user's body. Optionally, the attachment may be provided via a strap, and alternatively via a securing patch. Typically, the first sensor is connected to said control unit via connection means, typically comprising a connection wire, extending there between.

Alternatively, said first sensor is provided by said second electrode.

Preferably, a third electrode is provided between said first and second electrodes. Typically, said third electrode is adapted to act as a common node which allows an electrical current to be passed between said third electrode and said first electrode and selectively between said third electrode and said second electrode.

Preferably, said first, second and third electrodes are located on a band adapted to be placed adjacent specific muscles on the user such that each electrode is in communication with an area of skin adjacent those muscles in order to activate motor nerves thereof. More preferably, said band is adapted to be placed around a user's abdominal area such that each electrode is in communication with an area of skin adjacent the abdominal muscles.

Preferably, said control unit is adapted to selectively impart an electrical current between the or each electrode in order to cause selected nerve stimulation.

Preferably, said control unit is adapted to alternate between a stimulation mode where selected nerves and hence selected muscles are stimulated and a monitoring mode where the electrical activity of the user's heart is monitored. More preferably, when the stimulating mode is in operation, the monitoring mode is deactivated. Typically, the monitoring mode is deactivated by a blanking signal selectively emitted from said control unit when said stimulating mode is in operation.

Preferably, at least one electrode is adapted for use in a monitoring mode such that when said control unit is in the monitoring mode the or each electrode may be used in conjunction with said first sensor in order to monitor electrical activity of the user's heart.

Preferably, said control unit is adapted to substantially prevent electrical current produced as a result of the stimulation mode from entering the or each sensor. Typically, this is provided by providing an impedance to flow of current into the or each sensor which is likely to create a leakage current in the body and or to damage components of the control unit. Preferably, said impedance is provided by resistors and capacitors provided in the control circuit.

Preferably, said control unit is further adapted to substantially prevent electrical current being imparted from the or each sensor into the user's body. Typically, this is provided by providing between said control unit and each sensor, an impedance to flow of current entering or leaving said control unit.

Preferably, said control unit comprises a processor adapted to provide an analogue pulse signal. More preferably, said control unit further comprises a converter adapted to convert the analogue signal produced by said control unit into a digital pulse sequence.

Preferably, said control unit further comprises a pulse amplifier adapted to amplify said analogue pulse sequence.

Preferably, said control unit further comprises a current steering network adapted to selectively direct said amplified analogue pulse sequence to the or each electrode. Typically, said current steering network comprises an H-bridge arrangement of switching transistors.

Preferably, said control unit further comprises a differential amplifier adapted to amplify the signal obtained from the or each sensor.

Preferably, said control unit further comprises a band pass filter adapted to derive, as a series of pulses, the user's heart rate from said amplified sensor signal.

Preferably, said control unit further comprises a threshold detector adapted to filter electrical noise detected by the or each sensor. Preferably, said threshold detector automatically adjusts the threshold level depending upon the average signal amplitude detected over a given sample period.

Preferably, the apparatus further comprises a display unit in communication with said control unit, the display unit being capable of displaying relevant data to the user such as heart rate, duration of exercise etc. Optionally, the display unit comprises a hand held module connected to said control unit. Alternatively, the display unit comprises a discrete unit, such as a wristwatch, in communication with said control unit.

Preferably, the apparatus further comprises a command unit in communication with said control unit, the command unit being adapted to allow the user to modify the intensity of stimulation provided by said electrodes, the intended exercise duration etc. Optionally, the command unit comprises a hand held module connected to said control unit. Alternatively, the command unit comprises a discrete unit, such as a wristwatch, in communication with said control unit.

Preferably, the display unit and command unit are integrated into a single module.

Preferably, the control unit is further adapted to provide the user with a Quality Factor which represents the level of benefit from the exercise and is dependent upon the level of exertion during exercise wherein the control unit is further adapted to calculate the Quality Factor according to the following equation: —

QF=k₁T+k₂(c×cᵢ)

and where QF is the Quality Factor, k₁ is a weighting and scaling component for the cardiovascular component of the exercise, T is the time of exercise at or above a target heart rate, k₂ is a weighting and scaling factor for the electrical stimulation components of the exercise, c is the number of contractions and cᵢ is contraction intensity.

According to the present invention there is also provided a method of providing nerve stimulation and exercise monitoring comprising: —

applying an electrical current to selected motor nerves of a user's body via electrodes in a stimulation mode;

monitoring the electrical activity of the user's heart using at least a sensor in a monitoring mode, wherein during the monitoring mode the stimulation mode is deactivated.

Typically, the step of monitoring the electrical activity of the user's heart further comprises the step of detecting an electrocardiogram signal.

Preferably, the method of providing nerve stimulation and exercise monitoring further comprises the step of applying the electrical current to the selected motor nerves of selected muscles at a time of application corresponding to an optimised point in the electrocardiogram output signal. Preferably, the time of application is delayed from the time of detection of a wave, preferably the R wave, of the electrocardiogram signal on the or each sensor.

Preferably, the method of providing nerve stimulation and exercise monitoring further comprises the step of providing the user with a Quality Factor which represents the level of benefit from the exercise and is dependent upon the level of exertion during exercise.

Preferably, the Quality Factor is calculated according to the following equation: —

QF=k₁T+k₂(c×cᵢ)

where QF is the Quality Factor, k₁ is a weighting and scaling component for the cardiovascular component of the exercise, T is the time in minutes of exercise at or above a target heart rate, k₂ is a weighting and scaling factor for the electrical stimulation components of the exercise, c is the number of contractions per minute (set by the user) and cᵢ is contraction intensity (set by the user).

Typically, the Quality Factor is displayed to the user on a display device. Alternatively, the Quality Factor is relayed to the user via an audio device.

Embodiments of the present invention will now be described, by way of example only, with reference to the accompanying figures, in which: —

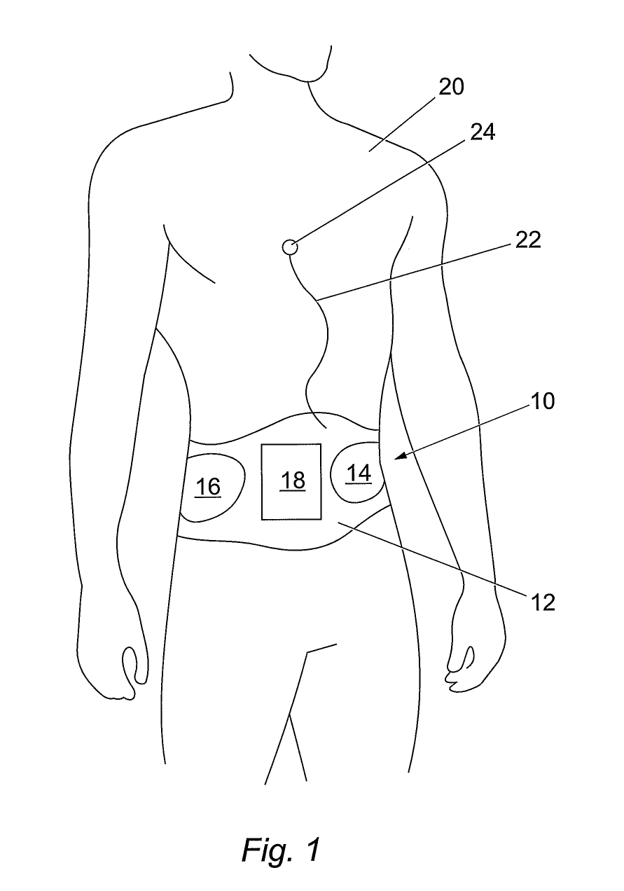

FIG. 1 is a schematic illustration of apparatus in accordance with the present invention as worn by a user during exercise (with sections cut away for clarity);

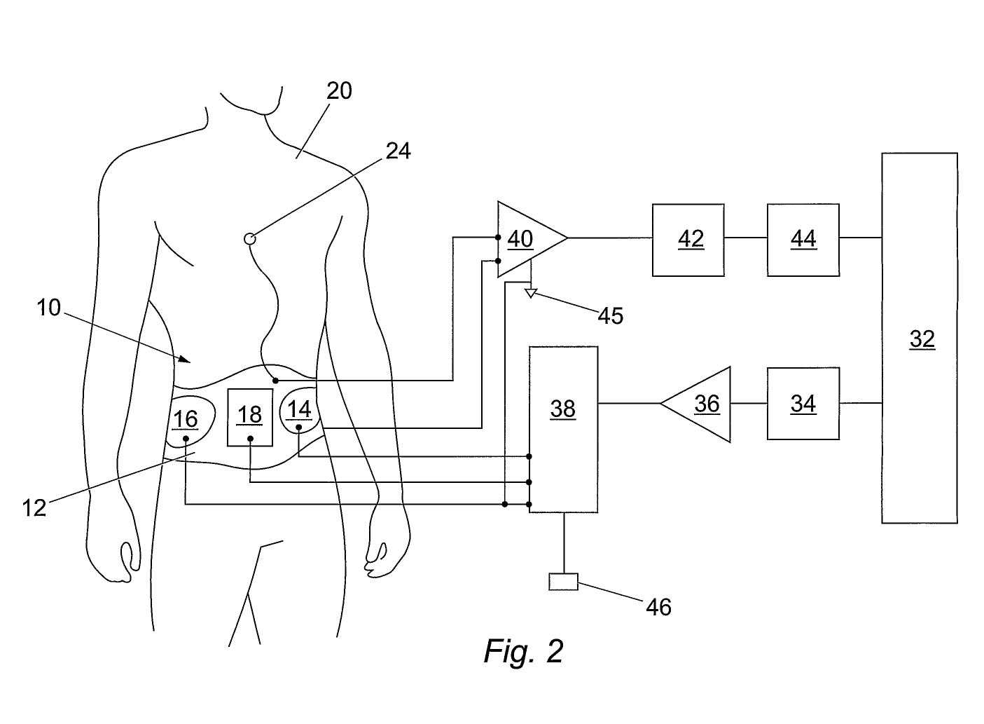

FIG. 2 is a schematic block diagram showing the components of the apparatus of FIG. 1; and

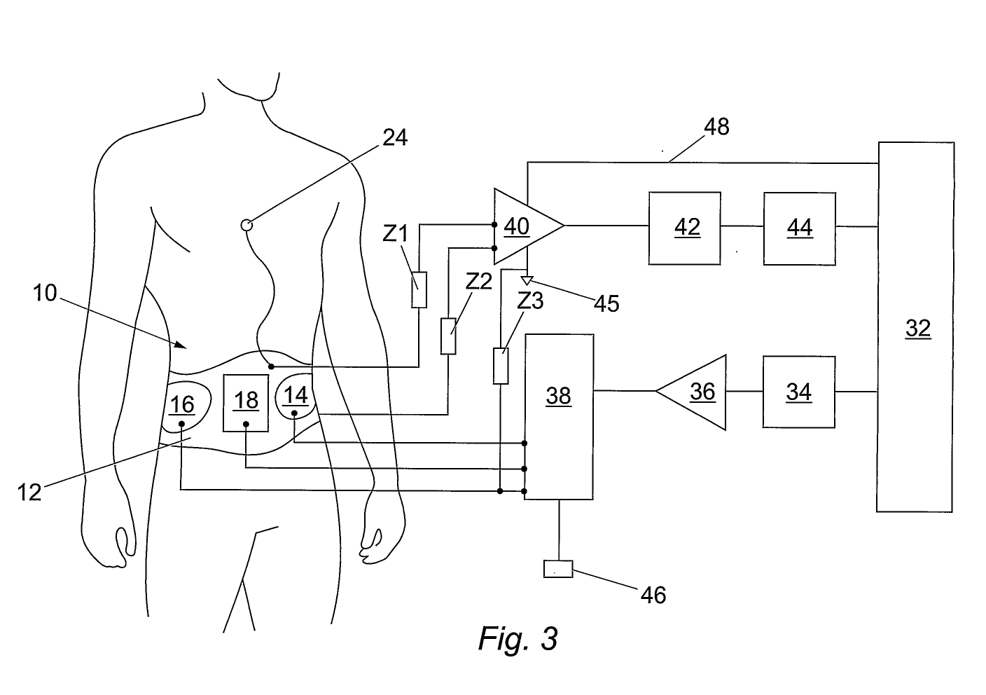

FIG. 3 is a schematic block diagram showing the components of the apparatus of FIG. 1 with additional protective circuits installed; and

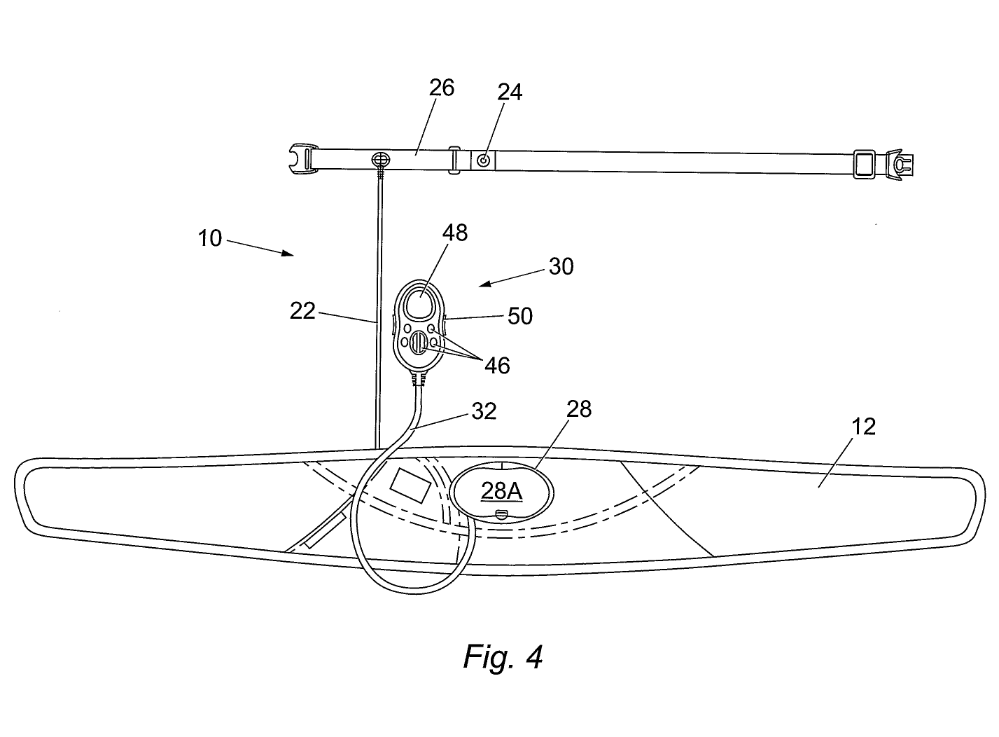

FIG. 4 is a more detailed illustration of the apparatus of FIG. 1 prior to use.

It should be noted that for the sake of clarity, the following description will refer to the location of components from the user's point of view when in use, that is components described as being on the left hand side of the apparatus relate to those components provided on the user's left hand side and components described as being on the user's right hand side relate to those components on the user's right hand side in use.

Referring to FIGS. 1 and 4, the apparatus 10 comprises an abdominal belt 12 having left electrode 14, right electrode 16 and central electrode 18 mounted on the inner circumference thereof such that the electrodes 14, 16 and 18 make contact with the user's 20 abdomen. A remote sensor 24 is also provided on the user's chest by a suitable attachment means such as an adjustable chest strap 26 (for a male user) as shown in FIG. 4 or a reusable patch, not shown, (for a female user). The remote sensor 24 is connected, via a lead 22, to a base unit 28 on the belt 12. The base unit 28 is provided with a control unit 30 which may be remote from the base unit and connected via a control lead 32.

The abdominal belt 12 is shaped to provide a comfortable fit around the abdomen of the user 20 when in use (for both male and female users) and may be made of any suitable material such as neoprene or other elastic material. The abdominal belt 12 is provided with a docking location which locates the base unit 28 on the belt 12.

The left electrode 14 and right electrode 16 are of typical construction and are each positioned along the band 12 in a position which ensures that when the band 12 is secured around the abdomen of the user 20, the electrodes 14 and 16 will contact the area of skin between the user's rib cage and pelvis on the left and right hand side of the user. The correct location of electrodes 14 and 16 is important since the abdominal muscle motor nerves are confined between the rib cage and the pelvis.

The central electrode 18 is located at the centre of the belt 12 such that when the belt 12 is attached to the abdomen the electrode 18 will contact the skin at the umbilicus. This allows further targeted stimulation of the central abdominal muscles. The central electrode 18 is similar in composition to the left and right electrodes 14 and 16 and provides a common node for the current from the left electrode 14 to pass when the left abdominal muscles are stimulated and for the current from the right electrode 16 to pass when the right abdominal muscles are stimulated. It will be understood by the skilled reader that in the following description muscles are actually stimulated by stimulating the motor nerve which supplies them.

Selected electrodes of the group of electrodes 14, 16 and 18 may be capable of detecting electrical signals in addition to producing electrical current, as will be discussed subsequently.

Remote sensor 24 comprises a sensor capable of detecting electrical signals produced by a heart beat and may be used in conjunction with at least one of the group of electrodes 14, 16 and 18 for this purpose.

The base unit 28 comprises a body 28A which houses the electrical components required to control the system. Referring to FIG. 2, the electrical components include a micro-controller 32, digital to analogue converter 34, pulse amplifier 36, current steering network 38, differential amplifier 40, band pass filter 42 and threshold detector 44. The interaction between these components will be described subsequently.

Control unit 30 is provided with control buttons 46 which allow the user to vary the intensity of the muscle stimulation provided by the apparatus 10 and to switch between operation modes etc. A display screen 48 allows the user to view information such as the intensity, duration of the exercise, and Quality Factor (discussed subsequently). A clip 50 is also provided on the rear of the unit 30 in order to allow the user to clip the base unit to the belt 12 (or other location) as desired. This frees the user's hands for use in the exercise being performed.

In use, the belt 12 of the apparatus 10 is attached to the user's abdomen such that electrodes 14, 16 and 18 on the belt contact the skin at the locations previously described. The remote sensor 24 is then attached to the chest via the chest strap 26 (for a male user) or the patch 25 (for a female user). If not already in place, the base unit 28 and control unit 30 are then attached to the belt 12. Once switched on, the apparatus 10 is ready for use.

When the user begins to exercise he/she can select a level of nerve (and hence muscle) stimulation using the control unit 30. Depending upon the mode and level of muscle stimulation selected, the micro-controller 32 initiates stimulation signals through the digital to analogue converter 34. Each analogue pulse of the signal from the converter 34 is amplified by pulse amplifier 36 which inputs the amplified pulses into current steering network 38. Current steering network 38 may typically comprise an H-bridge arrangement of switching transistor such as that used in motor drivers. The current steering network 38 directs the stimulation pulses to source at any one of the electrodes 14, 16 and 18 and to sink at any other of the electrodes depending upon the mode/intensity selected by the user 20. Stimulation pulses are returned to the power ground 46 of the system. This provides a route through which the electrical current may flow (through the user's abdominal muscles). It should be noted that although the previously described steering network is preferable, alternative arrangements are possible using, for example, transformers or relays.

When the pulse signal reaches, for example, the left electrode 14 a potential is created between electrode 14 and 18. The only route for the current to flow between electrode 14 and 18 is through the muscles of the user's abdomen and it is this flow of current which causes the abdominal muscles to contract (when current flows) and relax (when current does not flow). The cycle of contraction and relaxation can typically be varied as desired and is typically in the region of around 6 cycles per minute. A similar effect occurs on the abdominal muscles on the right hand side of the user's abdominal muscles when the pulse signal is sent to the right electrode 16.

This allows the user's abdominal muscles to be stimulated throughout the exercise regardless of the activity undertaken.

As the user 20 is exercising, the activity of his/her heart will increase (relative to resting heart activity) and it is desirable for the user to be able to monitor this. In the present embodiment, this is achieved using the remote sensor 24 in conjunction with the left abdominal electrode 14. It should be noted that, when used in this way, the electrode 14 may be regarded as a sensor 14 since it is used in a sensing mode. Similarly, right electrode 16 and central electrode 18 may be regarded as sensors if they are used in a sensing mode.

The location of the remote sensor 24 allows the activity of the heart to be monitored at the thoracic (chest) region. The measurement signal of this activity of the heart is commonly referred to as an “electrocardiogram” (ECG).

The electrical potential difference between sensor 24 and electrode 14 will be relatively high due to the relatively large difference in distances between the sensor 24 and electrode 14 from the source (the heart).

The location of the left electrode 14 allows the activity of the heart to be monitored at the abdominal region. The signal detected by the electrode 14 will be relatively weak when compared to that detected at the sensor 24. The signal at electrode 14 is weak since the electric dipole which produces the signal on the user's chest is located in the thoracic region and is significantly attenuated in the lower abdominal region.

The potential difference between remote sensor 24 and electrode 14 is a dynamic ECG signal representative of the cardiac cycle. The resulting ECG signal can be directly related to the heart rate of the user 20 by inputting the ECG signal into differential amplifier 40, which amplifies the signal, and then passing the amplified signal into band pass filter 42 which extracts the heart rate as a series of pulses. This signal is then passed to the micro-controller 32, via threshold detector 44, which displays the heart rate to the user on display 48. In this regard, the threshold detector 44 is preferably an automatic threshold level detector which automatically adjusts the threshold level in proportion to the average signal amplitude over a sample period, typically of a few seconds. This allows a useful signal to be recovered that is not drowned out by the electrical noise created by movement of the user 20 during the exercise routine.

In addition to using the left electrode 14 in conjunction with the remote sensor 24, the right electrode 16 and central electrode 18 can also be used as a reference electrode to reduce interference and inaccuracies in the measured ECG. This is particularly useful in reducing movement artefact which occurs due to the vertical accelerations of the body caused by walking and running.

It should be noted that although described separately above, when both the muscle stimulation mode and the heart rate mode are activated by the user 20, the micro-controller 32 rapidly switches between providing the muscle stimulation and monitoring the heart rate. This has the effect of apparently doing both tasks simultaneously. In this regard, the heart rate signal-monitored by the micro-processor 32 should not be affected by the electrical disturbance created by the muscle stimulation electrodes since it is timed to only monitor heart rate when no muscle stimulation is taking place.

This enables the user to monitor their heart rate in order to keep within a specific exercise zone. Such an exercise zone may be in the region of 60% to 70% of the theoretical maximum heart rate for a person of that age and fitness level. This allows the user to increase their work rate if the heart rate falls below the minimum recommended threshold and to decrease it if their heart rate goes above the maximum recommended threshold.

Although the micro-controller 32 is programmed to only monitor heart rate when the stimulation pulse is not being applied, the stimulation pulse will attempt to pass through the remote sensor 24 and any of the inactive electrodes back towards the differential amplifier 40. This would create an unintended leakage current and possibly damage the input of the amplifier 40 and protection against this should therefore be provided.

As shown in FIG. 3, protection against the above mentioned is provided by placing limiting resistors Z1 and Z2 before the differential amplifier 40 and resistor Z3 across the steering network output and the signal ground. These may be used in conjunction with voltage limiting devices such as zener diodes to protect against excessive voltages being applied to the amplifier 40 input. Choosing the value of resistors Z1, Z2 and Z3 involves considering the specific nature of the present system as discussed subsequently.

As will be appreciated by the reader, the stimulation provided by the electrodes on the band 12 causes a large electrical disturbance on the surface of the user's body and this must be accounted for in the detection of the heart rate in order to maintain accuracy.

Bio-amplifiers (amplifiers which amplify biological signals) are often AC coupled at their inputs since they must reject the DC potential (known as “half-cell potential”) component which occurs at the sensing electrodes. In order to achieve this without compromising low frequency response, a high value capacitor, often greater than 1 μF, is normally used. The problem with this solution is that high value capacitance coupled with the high resistance required to protect the amplifier 40 leads to long time constants for amplifier recovery following application of a stimulation pulse. This is because the AC coupling capacitor charges up during the stimulation pulse and must discharge before the amplifier 40 can continue operation. A compromise must therefore be reached, where a reasonable time constant is provided. This typically lies in the region somewhere between 0.25 seconds and 2 seconds. Placing these components before the amplifier 40 has a similar effect to preceding the amplifier 40 by a first order high pass filter. This results in a loss of some low frequency information in the ECG signal detected; however, this is acceptable in the present application since ECG signal fidelity is not particularly important and the principle measure of interest here is heart rate. The heart rate signal will be substantially unaffected by such a loss in ECG fidelity caused by the high pass filter. Taking a into consideration these factors, a suitable time constraint is provided using, for example, 5MΩ of amplifier input resistance and 47 nF of AC coupling capacitor.

A blanking signal represented by line 48 can also be selectively applied to the amplifier 40 to switch it off during stimulation pulses. A similar blanking signal (not shown) can be used to disable the threshold detector 44.

As well as protecting components of the apparatus 10 from damage it is important to ensure that the user 20 is protected from excessive and wayward electrical pulses. For this reason, safety standards specify that electrodes (such as sensor 24) which are intended to only detect electrical signals (FCG signals) must not be allowed to deliver stimulation pulses. Such unintentional current is know as an auxiliary current and, if allowed, could be dangerous to the user due to the sensor 24 proximity to the user's heart.

Various switches such as transistors and relays may be placed in the control circuit in order to both protect components of the circuit and to protect the user 20; however, such components can fail in extreme conditions and it is therefore necessary to ensure further protection to ensure that the device “fails safe”.

Preventing leakage of auxiliary current requires that the input resistance to the amplifier 40 is both high and capable of withstanding high input voltages. The amplifier 40 will have an inherent resistance; however, it is not sufficient to rely on this resistance since it may break down with high voltages. It is therefore safest to place an additional high resistance resistor, in the order of 100 kΩ, in series on each lead connecting the electrodes 14, 16 and the sensor 24 to the micro-controller 32. These resistors are represented on FIG. 3 by Z1, Z2 and Z3 respectively.

Accordingly, the invention described provides exercise equipment which allows the user to actively monitor their heart rate and muscle stimulation to individual preference without the risk of being subjected to uncontrolled electrical pulses. The invention therefore allows the user to simultaneously perform two different forms of exercise at any one time; namely, Electrical Muscle Stimulation and cardiovascular exercise with heart rate monitoring. Electrical Muscle Stimulation does not, by itself, create a significant load to the cardiovascular system and therefore has little or no exercise benefit to that system. It does however load and thereby promote performance improvement in the target muscle.

In cardiovascular exercise, the value of an exercise session can be estimated by the amount of time the user spends at or above the target heart rate. For resistance training on the other hand, time is not important and instead it is the number of repetitions of muscle contraction under load that is important.

The present invention provides for the calculation and display of an exercise Quality Factor which is based on the amount of time the user spends at or above the target heart rate and the number of stimulated muscle contractions completed.

The resultant Quality Factor is displayed to the user (either on the display device or by an audio device) and is updated as the user exercises. The Quality Factor is calculated according to the following equation: —

QF=k₁T+k₂(c×cᵢ)

Where QF is the Quality Factor, k₁ is a weighting and scaling component for the cardiovascular component of the exercise, T is the time in minutes of exercise at or above a target heart rate, k₂ is a weighting and scaling factor for the electrical stimulation components of the exercise c is the number of contractions per minute (set by the user) and cᵢ is contraction intensity (set by the user).

The contraction intensity is changed by altering the flow of current between the electrodes and can be altered by the user to a desired level, or as in the following example, may be set to 1.

A typical value for k1 would be k1= 1/15 and for k2 would be k2= 1/180 although obviously other values could also be chosen, depending on the precision of the display device and the relative importance of each component of the exercise. With the above values, a 60 minute walking or jogging exercise at the target heart rate, with 3 maximal muscle contractions per minute would yield a Quality Factor of 5 according to the following: —

$$QF = \frac{1}{15} \times 60 + \frac{1}{180} ( 3 \times 60 \times 1 ) = 5$$

The combination of Electrical Muscle stimulation and cardiac monitoring in the present invention also has the advantage of allowing the electrical stimulus provided to desired muscles to coincide with a defined point in the cardiac cycle. For example, the delivery of the stimulus may be timed to occur a fixed time before the R wave of the ECG. The extent of the time delay can be adjusted to improve the efficacy of the assisted venous return.

Modifications and improvements may be made to the foregoing without departing from the scope of the invention, for example:

The micro-controller 32 can be programmed to provide a personalised muscle stimulation sequence which may be saved in the memory of the micro-controller 32. This could also automatically adapt the stimulation cycle to take account of the heart rate detected, that is, if the detected heart rate is high, the stimulation provided by the band 12 would automatically increase in order to increase the intensity of the overall exercise and vice versa. Alternatively, the micro-controller 32 may be programmed to decrease the intensity of the stimulation provided by the electrodes on the band 12 as the heart rate increases in order to focus training on the cardiovascular exercise as opposed to the abdominal stimulation and vice versa.

The abdominal band 12 could be replaced by or complemented with other groups of stimulation electrodes such as thigh and buttock electrodes.

The control unit 30 may be incorporated into a wristwatch or other separate unit which uses a wireless communication to transmit and receive information to and from the base unit 28.

The preferred embodiment described above employs a sensor 24 located close to the user's heart in conjunction with a sensor 14 located at the abdominal region (provided for instance by electrode 14) in order to detect the electrical potential there between (and hence the heart rate of the user); however, alternatively the electrical potential is detected between two abdominal sensors (provided for instance by electrodes 14 and 16) or, in a further alternative, two sensors may be provided on the chest strap 26/patch (not shown) and two separate abdominal electrodes provided for muscle stimulation.

Although, primarily described in relation to muscle stimulation, the nerve stimulation apparatus described may alternatively be used for stimulation of nerves for pain relief or massage.

1. Nerve stimulation apparatus comprising: —
a control unit;
a first electrode;
a second electrode, said first and second electrodes being adapted to impart an electrical current there between;
a first sensor; and
a second sensor, said first and second sensors being adapted to detect an electrical signal differential detected there between.

2. Nerve stimulation apparatus according to claim 1, wherein said first and second electrodes are adapted to provide electrical stimulation of a motor nerve of a user's muscles located adjacent to said first and second electrodes.

3. Nerve stimulation apparatus according to claim 1, wherein said first and second electrodes are provided adjacent the user's abdominal muscles.

4. Nerve stimulation apparatus according to claim 1, wherein said first and second electrodes are provided at a location close to a peripheral motor nerve.

5. Nerve stimulation apparatus according to claim 1, wherein said first and second sensors are adapted to monitor a user's heart rate.

6. Nerve stimulation apparatus according to claim 1, wherein said second sensor is provided by said first electrode.

7. Nerve stimulation apparatus according to claim 1, wherein a third sensor is provided by said second electrode.

8. Nerve stimulation apparatus according to claim 7, wherein said first sensor is adapted to be provided at a location that is closer to the user's heart than the or each of the second and third sensors.

9. Nerve stimulation apparatus according to claim 1, wherein the first sensor is adapted to be attached to the thoracic region of a user's body by attachment means.

10. Nerve stimulation apparatus according to claim 9, wherein the attachment means comprises a strap or securing patch.

11. Nerve stimulation apparatus according to claim 1, wherein the first sensor is connected to said control unit via connection means.

12. Nerve stimulation apparatus according to claim 1, wherein said first sensor is provided by said second electrode.

13. Nerve stimulation apparatus according to claim 1, wherein a third electrode is provided between said first and second electrodes.

14. Nerve stimulation apparatus according to claim 13, wherein said third electrode is adapted to act as a common node which allows an electrical current to be passed between said third electrode and said first electrode and selectively between said third electrode and said second electrode.

15. Nerve stimulation apparatus according to claim 13, wherein said first, second and third electrodes are located on a band adapted to be placed adjacent specific muscles on the user such that each electrode is in communication with an area of skin adjacent those muscles in order to activate motor nerves thereof.

16. Nerve stimulation apparatus according to claim 15, wherein said band is adapted to be placed around a user's abdominal area such that each electrode is in communication with an area of skin adjacent the abdominal muscles.

17. Nerve stimulation apparatus according to claim 1, wherein said control unit is adapted to selectively impart an electrical current between the or each electrode in order to cause selected nerve stimulation.

18. Nerve stimulation apparatus according to claim 17, wherein said control unit is adapted to alternate between a stimulation mode where selected nerves and hence selected muscles are stimulated and a monitoring mode where the electrical activity of the user's heart is monitored wherein when the stimulating mode is in operation, the monitoring mode is deactivated.

19. Nerve stimulation apparatus according to claim 18, wherein the monitoring mode is deactivated by a blanking signal selectively emitted from said control unit when said stimulating mode is in operation.

20. Nerve stimulation apparatus according to claim 18, wherein at least an electrode is adapted for use in a monitoring mode such that when said control unit is in the monitoring mode the or each electrode may be used in conjunction with said first sensor in order to monitor electrical activity of the user's heart.

21. Nerve stimulation apparatus according to claim 18, wherein said control unit is provided with safety means to substantially prevent electrical current produced as a result of the stimulation mode from entering the or each sensor.

22. Nerve stimulation apparatus according to claim 21, wherein the safety means is provided by an impedance to flow of current into the or each sensor which is likely to create a leakage current in the body and/or to damage components of the control unit.

23. Nerve stimulation apparatus according to claim 21, wherein said control unit is provided with further safety means to substantially prevent electrical current being imparted from the or each sensor into the user's body.

24. Nerve stimulation apparatus according to claim 23, wherein the further safety means is provided by providing between said control unit and each sensor an impedance to flow of current entering or leaving said control unit.

25. Nerve stimulation apparatus according to claim 1, wherein said control unit comprises a processor adapted to provide an analogue pulse signal and a converter adapted to convert the analogue signal produced by said control unit into a digital pulse sequence.

26. Nerve stimulation apparatus according to claim 25, wherein said control unit further comprises a pulse amplifier adapted to amplify said analogue pulse sequence.

27. Nerve stimulation apparatus according to claim 26, wherein said control unit further comprises an H-bridge arrangement of switching transistors to provide a current steering network adapted to selectively direct said amplified analogue pulse sequence to the or each electrode.

28. Nerve stimulation apparatus according to claim 27, wherein said control unit further comprises a differential amplifier adapted to amplify the signal obtained from the or each sensor.

29. Nerve stimulation apparatus according to claim 28, wherein said control unit further comprises a band pass filter adapted to derive, as a series of pulses, the user's heart rate from said amplified sensor signal.

30. Nerve stimulation apparatus according to claim 29, wherein said control unit further comprises a threshold detector adapted to filter electrical noise detected by the or each sensor.

31. Nerve stimulation apparatus according to claim 30, wherein said threshold detector automatically adjusts the threshold level depending upon the average signal amplitude detected over a given sample period.

32. Nerve stimulation apparatus according to claim 1, wherein the apparatus further comprises a display unit in communication with said control unit, the display unit being capable of displaying relevant data to the user such as heart rate, duration of exercise.

33. Nerve stimulation apparatus according to claim 1 further comprising a command unit in communication with said control unit, the command unit being adapted to allow the user to modify the variables such as the intensity of stimulation provided by said electrodes and the intended exercise duration.

34. Nerve stimulation apparatus according to claim 32, wherein the display unit and command unit are integrated into a module in communication with said control unit and adapted to be hand held or located on a wristwatch.

35. Nerve stimulation apparatus according to claim 1, wherein the control unit is adapted to provide the user with a Quality Factor which represents the level of benefit from the exercise and is dependent upon the level of exertion during exercise wherein the control unit is further adapted to calculate the Quality Factor according to the following equation: —
QF=k₁T+k₂(c×cᵢ)
and where QF is the Quality Factor, k₁ is a weighting and scaling component for the cardiovascular component of the exercise, T is the time of exercise at or above a target heart rate, k₂ is a weighting and scaling factor for the electrical stimulation components of the exercise, c is the number of contractions and cᵢ is contraction intensity.

36. A method of providing nerve stimulation and exercise monitoring comprising: —
applying an electrical current to selected motor nerves of a user's body via electrodes in a stimulation mode;
monitoring the electrical activity of the user's heart using at least a sensor in a monitoring mode, wherein during the monitoring mode the stimulation mode is deactivated.

37. A method according to claim 36, wherein the step of monitoring the electrical activity of the user's heart further comprises the step of detecting an electrocardiogram signal.

38. A method according to claim 36, further comprising the step of applying the electrical current to the selected motor nerves of selected muscles at a time of application corresponding to an optimised point in the electrocardiogram output signal.

39. A method according to claim 38, wherein the time of application is delayed from the time of detection of a wave of the electrocardiogram signal on the or each sensor.

40. A method according to claim 39, wherein the wave of the electrocardiogram signal is an R wave.

41. A method according to claim 36 further comprising the step of providing the user with a Quality Factor which represents the level of benefit from the exercise and is dependent upon the level of exertion during exercise wherein the Quality Factor is calculated according to the following equation: —
QF=k₁T+k₂(c×cᵢ)
and where QF is the Quality Factor, k₁ is a weighting and scaling component for the cardiovascular component of the exercise, T is the time of exercise at or above a target heart rate, k₂ is a weighting and scaling factor for the electrical stimulation components of the exercise, c is the number of contractions and cᵢ is contraction intensity.

42. A method according to claim 41, wherein the Quality Factor is displayed to the user on a display device or an audio device.
